(19) United States
(12) Patent Application Publication
(10) Pub. No.: US 2025/0111517 A1
(43) Pub. Date: Apr. 3, 2025

(54) SEGMENTATION OF A REGION OF INTEREST IN A MEDICAL IMAGE

(71) Applicant: Shanghai United Imaging Intelligence Co., Ltd., Shanghai (CN)

(72) Inventors: Wenzhe Cui, Cambridge, MA (US); Meng Zheng, Cambridge, MA (US); Arun Innanje, Lexington, MA (US); Ziyan Wu, Lexington, MA (US); Terrence Chen, Lexington, MA (US)

(73) Assignee: Shanghai United Imaging Intelligence Co., Ltd., Shanghai (CN)

(21) Appl. No.: 18/374,473

(22) Filed: Sep. 28, 2023

(51) Int. Cl. G06T 7/12; G06T 7/13; G06V 10/25; G16H 30/40

(52) U.S. Cl. CPC G06T 7/12; G06T 7/13; G06V 10/25; G16H 30/40; G06T 2207/20104

(57) ABSTRACT

An apparatus for annotating a medical image may be configured to obtain, automatically, an outline of a region of interest (ROI) in the medical image and determine, based on one or more inner control points and one or more outer control points. The one or more inner control points may be located within the ROI and the one or more outer control points may be located outside of the ROI. The outline may be subsequently adjusted based on a user input and the adjusted outline may be used to generate a segmentation of the ROI.

### BACKGROUND

Image segmentation may be used in digital image processing and analysis to identify parts or regions of interest in a medical image. For example, image segmentation may detect pixels or voxels of the medical image that may be associated with a region of interest, such as a patient's brain or some other organ of the patient. Traditional methods of medical image segmentation require manual editing by a person in order to accurately identify the actual region of interest in the medical image. Such manual editing may be a time-consuming and therefore costly task. Additionally, when different manual image segmentation methods are being used or different human annotators are being involved (e.g., for single image segmentation and segmentation across multiple images), it may be difficult to make sure that the segmentation results are consistent with each other (e.g., a similar contour for a same region of interest of a patient in each of the medical images).

### SUMMARY

Described herein are systems, methods and instrumentalities for segmentation of a region of interest (ROI) in a medical image. An apparatus configured to perform the segmentation task may obtain an outline of the ROI based on one or more control points located within the ROI or outside of the ROI. The apparatus may adjust the outline of the ROI based on a user input, wherein the user input may indicate a selection (e.g., of an area or a spot) within the outline or outside of the outline, or a change to a distance between the outline and the one or more control points. Based on the adjusted outline of the ROI, the apparatus may determine a segmentation of the ROI.

In examples, the apparatus may determine the outline of the ROI in the medical image by determining multiple anchor points associated with the ROI, and further determining, based on the multiple anchor points, one or more inner control points located within the ROI and one or more outer control points located outside of the ROI. The apparatus may then obtain the outline of the ROI based on the one or more inner control points and the one or more outer control points. The apparatus may determine the multiple anchor points associated with the ROI based on a segmentation mask associated with the ROI, wherein the segmentation mask may be generated based on human annotation or a machine learning model. In examples, the apparatus may determine the one or more inner control points and the one or more outer control points by determine a connecting line that runs through a pair of anchor points, select a point on the connecting line that is within the ROI as an inner control point, and select a point on the connecting line that is outside of the ROI as an outer control point. In examples, the outline of the ROI may be located between the one or more inner control points and the one or more outer control points, and the apparatus may determine the outline based on a predetermined value that indicates a proximity of the outline to the one or more inner control points or the one or more outer control points.

In examples, adjusting the outline of the ROI based on the user input may include determining whether the selection (e.g., of an area or spot) indicated by the user input is within the outline or outside of the outline. If the determination is that the selection is within the outline, the outline may be adjusted to exclude an area corresponding to the selection. Conversely, if the determination is that the selection is outside of the outline, the outline may be adjusted to include an area corresponding to the selection.

In examples, the apparatus may provide a graphical user interface (GUI) element for changing an area surrounded by the outline. In these examples, adjusting the outline based on the user input may include receiving the user input via the GUI element, determining a value of the area surrounded by the outline based on the user input, and adjusting the outline of the ROI based on the determined value.

In examples, adjusting the outline based on the user input may include providing a preview of the adjustment to be made to the outline and adjusting the outline in response to receiving a confirmation of the adjustment.

### BRIEF DESCRIPTION OF THE DRAWINGS

A more detailed understanding of the examples disclosed herein may be had from the following description, given by way of example in conjunction with the accompanying drawings.

FIGS. 1A-1D illustrate examples of annotating a region of interest (ROI) in a medical image with inner control points and outer control point, according to embodiments described herein.

FIGS. 2A and 2B illustrate examples of adjusting the outline of an ROI based on a user input, according to embodiments described herein.

FIGS. 3A-3D illustrate additional examples of adjusting the outline of an ROI based on a user input, according to embodiments described herein.

FIGS. 4A-4C show illustrate additional examples of adjusting the outline of an ROI based on a user input, according to embodiments described herein.

FIG. 5 illustrates an example method for automatic image segmentation of a medical image, according to embodiments described herein.

FIG. 6 illustrates an example method for generating inner control points and outer control points, according to embodiments described herein.

FIG. 7 illustrates an example method for receiving user inputs via a graphical user interface and adjusting an outline of an ROI based on the user inputs, according to embodiments described herein.

FIG. 8 illustrates an example apparatus for performing the tasks described with respect to some of the embodiments disclosed herein.

### DETAILED DESCRIPTION

The present disclosure is illustrated by way of example, and not by way of limitation, in the figures of the accompanying drawings. A detailed description of illustrative embodiments will now be provided with reference to these figures. Although these embodiments may be described with certain technical details, it should be noted that the details are not intended to limit the scope of the disclosure. Further, while some embodiments may be provided in the context of medical image segmentation or annotation, those skilled in the art will understand that the techniques disclosed in those embodiments may be applicable to all types of images.

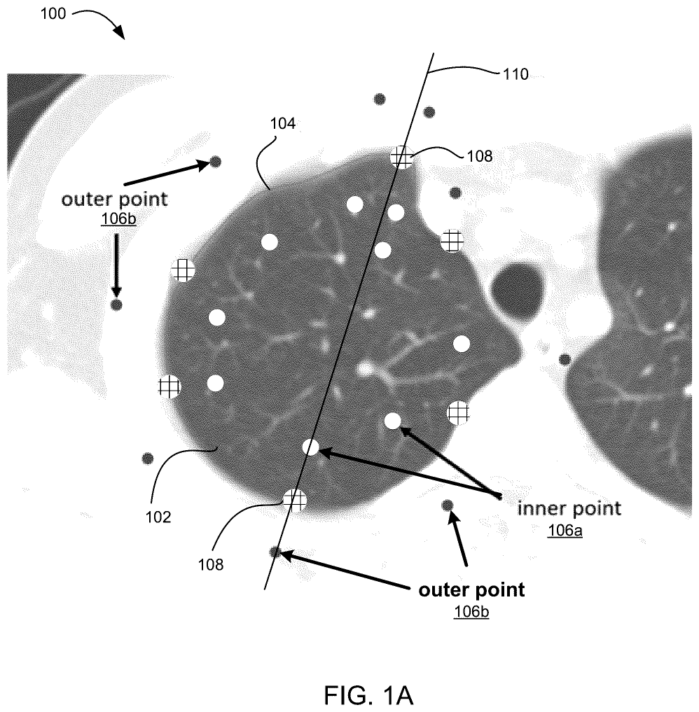

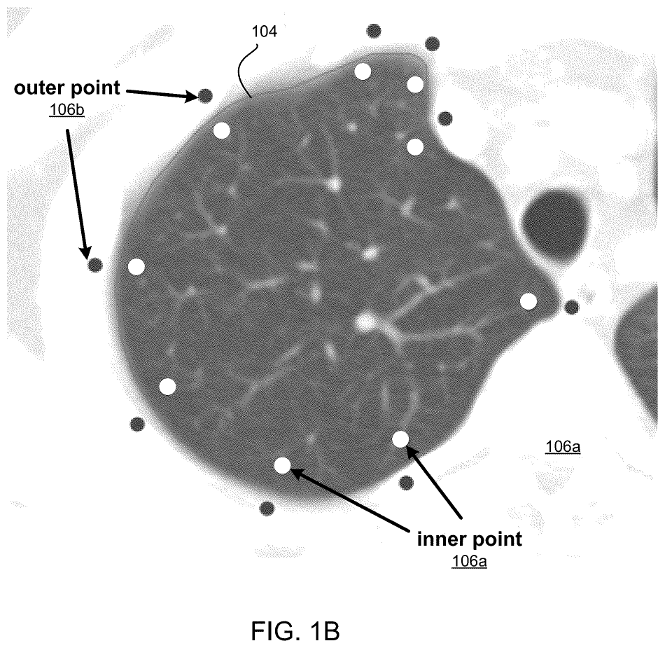

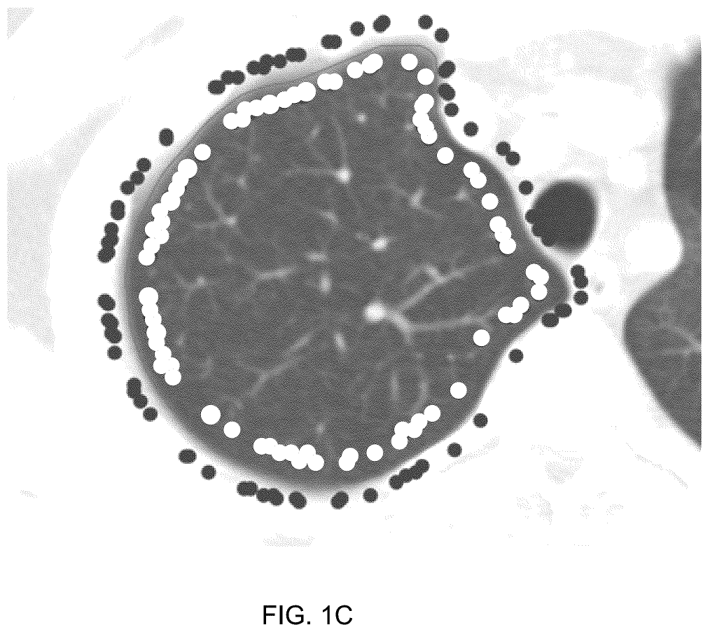

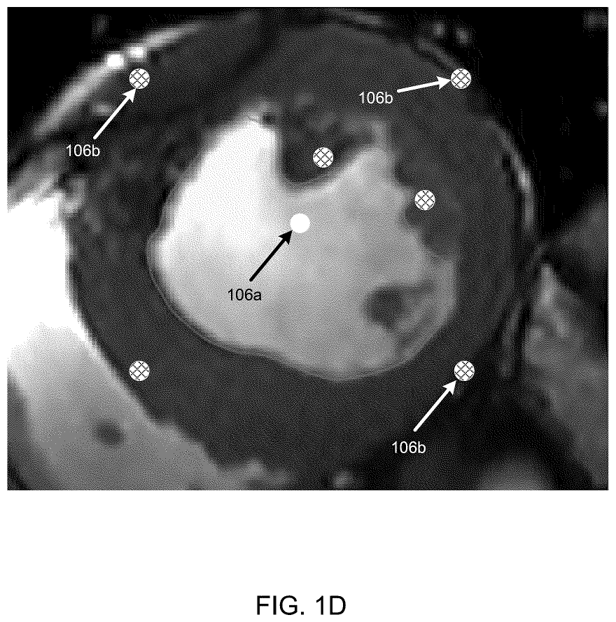

FIGS. 1A-1C show a medical image 100 including a region of interest (ROI) 102. The medical image 100 may be a scan image of a patient such as a scan image of the patient's brain, and the ROI 102 may be a portion of the patient's brain that may be the target of a medical investigation (e.g., for a medical abnormality). As shown in the example of FIG. 1A, an apparatus (e.g., referred to herein as an image segmentation apparatus) may be configured to obtain an outline 104 of the ROI 102 based on a plurality of control points, and further adjust (e.g., refine) the outline 104 based on a user input. In other examples (e.g., not shown in FIG. 1A), the apparatus may be configured to obtain the outline 104 of the ROI 102 based on a bounding box drawn around the ROI 102 (e.g., manually by a user or automatically by a computer program), and further adjust (e.g., refine) the outline 104 based on a user input. From the original or adjusted outline, the image segmentation apparatus may further determine (e.g., generate) a segmentation mask for the ROI 102, for example, by marking each point (e.g., pixel or voxel) within the outline 104 as belonging to the ROI 102 and each point outside of the outline 104 as not belonging to the ROI 102 (e.g., as belonging to a background). The segmentation mask may be generated with a color-coding (e.g., including a grayscale color-coding) to distinguish the portion of the medical image 100 corresponding to the ROI 102 from the rest of the medical image 100.

The control points used to obtain the outline 104 may include one or more inner control points 106a (e.g., the white-colored circles in FIG. 1A) located within the ROI 102 of the image 100. The control points may also include one or more outer control points 106b (e.g., the dark-colored circles in FIG. 1A) located outside of the ROI 102 of the medical image 100. The image segmentation apparatus may derive the one or more inner control points 106a and the one or more outer control points 106b based on multiple anchor points 108 (e.g., the pattern-filled circles in FIG. 1A) that may be obtained from a pre-determined segmentation mask of the ROI 102. For example, the medical image 100 may be part of a medical image series comprising the ROI 102, and the image segmentation apparatus may receive, via cross-plane propagation, a segmentation mask of the ROI 102 based on a previous medical image. Such a segmentation mask may be generated via human annotation or based on a machine learning model, and the image segmentation apparatus may select one or more points on the contour of the segmentation mask as the anchor points 108. Using these anchor points, the image segmentation apparatus may determine a connecting line 110 that runs through a pair of anchor points 108, and further select a point on the connecting line 110 located within the ROI 102 as an inner control point 106a. Similarly, the image segmentation apparatus may select a point on the connecting line 110 located outside of the ROI 102 as an outer control point 106b. The anchor points may be determined using various algorithms such as, e.g., the Ramer-Douglas-Peucker algorithm.

Once the inner control points 106a and the outer control points 106b are determined, the image segmentation apparatus may determine the outline 104 of the ROI 102 based on the inner control points 106a and the outer control points 106b. For example, the image segmentation apparatus may generate the outline in accordance with a preconfigured value that indicates a proximity of the outline 104 to the one or more inner control points 106a or the one or more outer control points 106b, such that the outline 104 may be located between the one or more inner control points 106a and the one or more outer control points 106b. As another example, the image segmentation apparatus may generate the outline using a machine-learning (ML) model trained for receiving the inner control points 106a and/or the outer control points 106b as inputs, and creating the outline based on the features of the target image and the inner/outer control points.

The respective locations of the one or more inner control points 106a and the one or more outer control points 106b may be controllable (e.g., with respect to how close the control points are to the outline of the ROI 102), as illustrated by FIG. 1B. For example, the image segmentation apparatus may receive from a user (e.g., via a user interface as explained below) an indication of at least one of a proximity (e.g., within 1 cm) of the one or more inner control points 106a to the outline 104 of the ROI 102, or a proximity of the one or more outer control points 106b to the outline 104 of the ROI 102. In response, the image segmentation apparatus may determine the one or more inner control points 106a and the one or more outer control points 106b based on the proximity indication.

The number of inner control points 106a and/or outer control points 106b generated by the image segmentation apparatus may also be controllable, as illustrated by FIG. 1C. For example, the image segmentation apparatus may receive (e.g., via the user interface described herein) an indication of at least one of a number of inner control points 106a to be generated or a number of outer control points 106b to be generated. The image segmentation apparatus may then determine the one or more inner control points 106a and the one or more outer control points 106b based on the indication of the respective numerical quantities for the inner and outer control points. It should be noted herein that the number of inner control points and/or the number of outer control points may be equal to or greater than one. For example, FIG. 1D illustrates an example in which the one or more inner control points may include a single inner control point 106a (e.g., the white-colored circles in FIG. 1D) and the one or more outer control points may include multiple outer control points 106b (e.g., the pattern-filled circles in FIG. 1D). Conversely, there may be examples where the one or more outer control points may include a single outer control point and the one or more inner control points may include multiple inner control points.

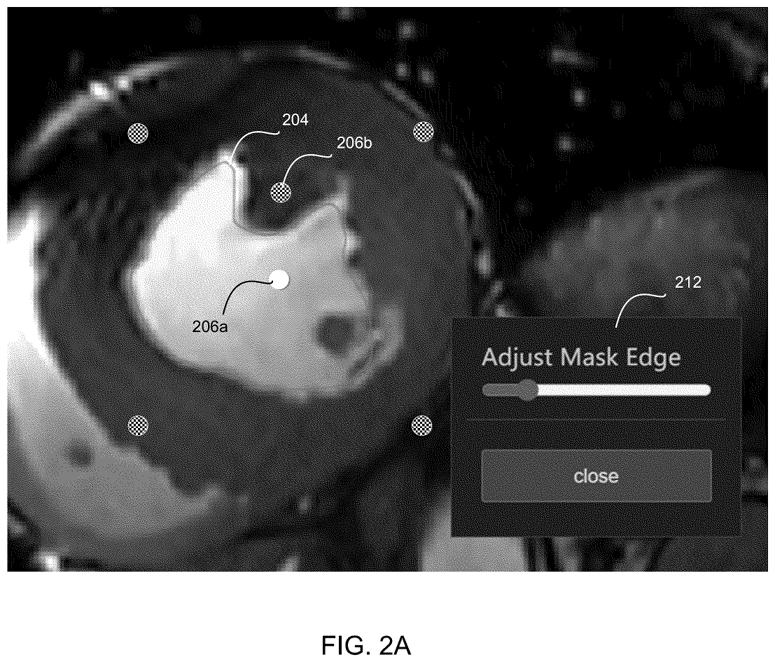

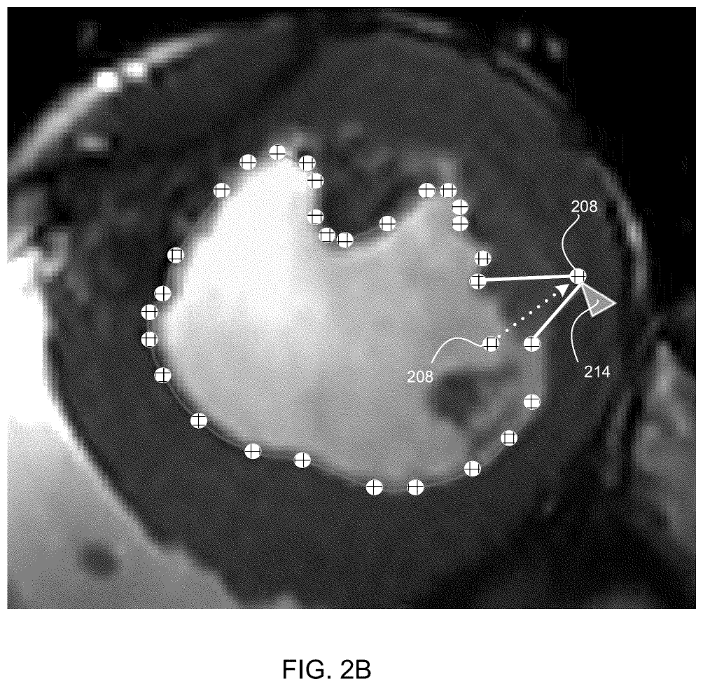

The outline 104 generated based on the control points (e.g., inner and outer control points) or the bounding box described herein may be adjusted or refined by a user, and the image segmentation apparatus may provide various tools to facilitate the adjustment. FIG. 2A illustrates an example of adjusting an outline 204 of an ROI (e.g., the outline 104 of ROI 102 shown in FIG. 1A) to change the area enclosed within the outline 204 (e.g., by expanding or shrinking the outline 204). As shown in FIG. 2A, the image segmentation apparatus described herein may receive a user input that may indicate a value (e.g., a threshold value) of the area surrounded by the outline 204. The user input may be received via a control element 212 (e.g., a slider) of a graphical user interface (GUI) provided by the image segmentation apparatus. In response to receiving the user input, the image segmentation apparatus may adjust the outline 204 based on the value indicated by the user input, for example, by stretching or shrinking the outline 204 to respectively include a larger area or a smaller area within the outline.

FIG. 2B illustrates an example of adjusting the outline 204 by adjusting the position of a point 208 on the outline. As shown in FIG. 2B, the image segmentation apparatus described herein may provide, on a graphical user interface (GUI), a presentation of the outline 204 that may be generated based on the inner and outer control points described herein. The image segmentation apparatus may subsequently receive a user input via the GUI (e.g., via the movement of a cursor 214) that indicates the movement of the point 208 from a first location to a second location. The image segmentation apparatus may then adjust the outline 204 based on the received user input, for example, by stretching the outline 204 in order to include the new position of the changed point 208.

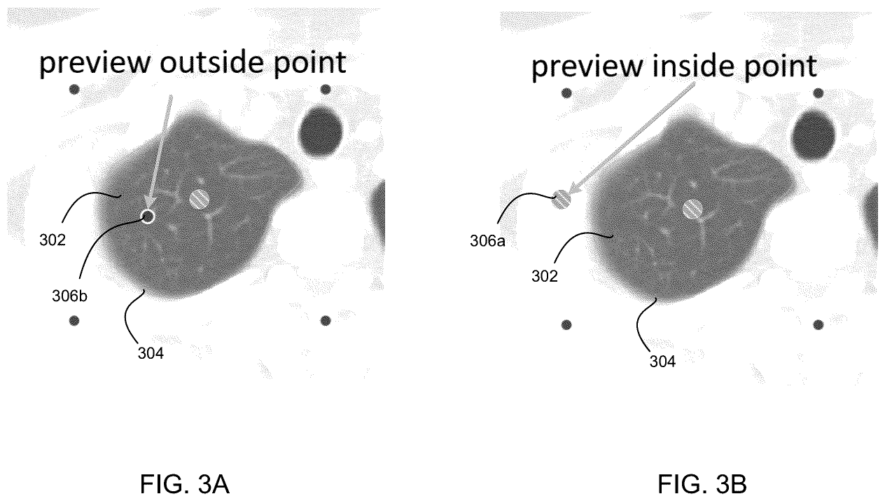

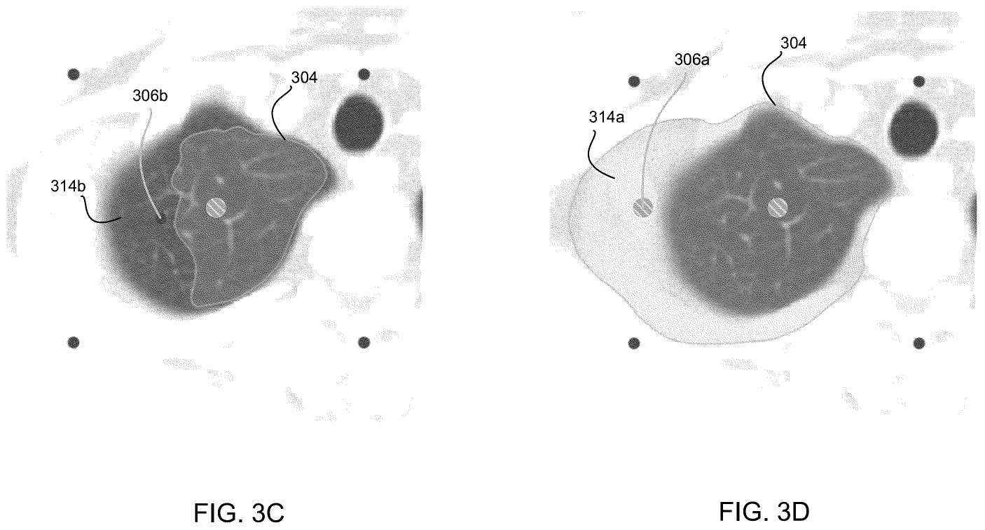

FIGS. 3A-3D illustrates another example technique for adjusting the outline 304 of an ROI 302, such as the outline 104 of FIG. 1A or the outline 204 of FIG. 2A. As shown in FIGS. 3A-3D, the image segmentation apparatus described herein may adjust the existing outline 304 based on a user input (e.g., received via a GUI provided by the image segmentation apparatus) that indicates the selection of an area (or a spot) within the present ROI 302 (e.g., as enclosed by the existing outline 304) or outside of the present ROI 302. If the selection is within the present ROI 302, as illustrated by FIG. 3A, the image segmentation apparatus may treat the selected spot as an additional outer control point 306b (e.g., a seed point), and may adjust the existing outline 304 (e.g., shrink the outline 304) to remove or exclude an area 314b that corresponds to the additional outer control point 306b, as illustrated by FIG. 3C. The adjusted outline 304 (e.g., the area 314b removed from the outline 304) may be calculated based on the updated set of outer control points including the additional outer control point 306b, for example, using the technique described herein. On the other hand, if the selection indicated by the user input is outside of the present ROI 302, as illustrated by FIG. 3B, the image segmentation apparatus may treat the selected spot as an additional inner control point 306a (e.g., a seed point), and may adjust the existing outline 304 (e.g., expand the outline 304) to add or include an area 314a that corresponds to the additional inner control point 306a, as illustrated by FIG. 3D. The adjusted outline 304 (e.g., the area 314a added to the outline 304) may be calculated based on the updated set of inner control points including the additional inner control point 306a, for example, using the technique described herein.

The procedure described with respect to FIGS. 3A-3D may reduce the number of user inputs (e.g., mouse clicks) required for adjusting (e.g., editing) an existing outline of an ROI. The operations involved in the procedure may be intuitive for various types of computing devices including, for example, touch screen devices, where a user may add and remove parts of a segmentation, outline, or contour by tapping on an area or spot within or outside of the ROI. In examples, the image segmentation apparatus described herein may be configured to present the additional inner control point 306a and outer control point 306b in different styles (e.g., different colors) such that a user may be made aware of the operations that may be triggered by their action. In examples, the image segmentation apparatus may be configured to provide a preview of the adjustment to be made to the existing outline 304 (e.g., in real time in accordance with the user's current mouse or finger position) and wait for a confirmation from the user before performing the adjustment (e.g., this may optimize the workflow and reduce the number of unnecessary taps or mouse clicks). In examples, when the user is in an outline editing mode and maintains a cursor (or a finger in the case of a touch screen device) at the same location for a certain time duration, the image segmentation apparatus may represent the to-be-adjusted outline with a dashed line to give the user a preview of the to-be-adjusted outline.

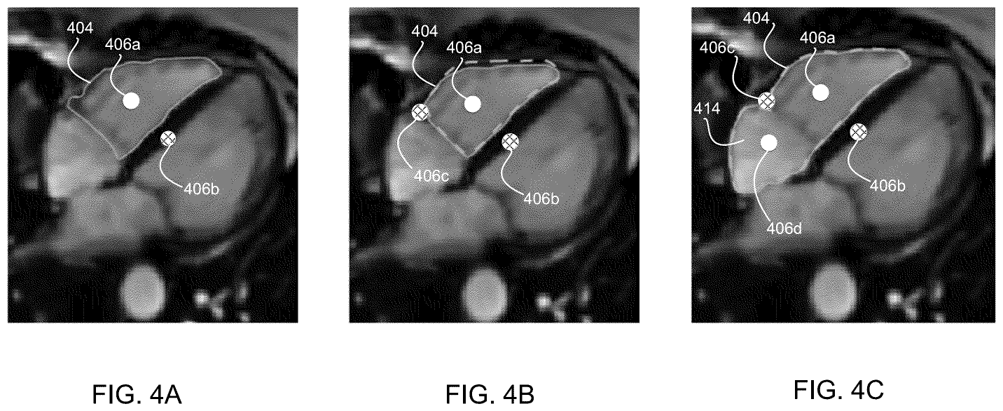

FIGS. 4A-4C illustrate additional examples of outline adjustments using the techniques described herein (e.g., via single mouse clicks). FIG. 4A shows an example of an initial outline 404 that may be generated based on an inner control point 406a and an outer control point 406b. FIG. 4B illustrates an example of adjusting the outline 404 to remove a corner area of the outline 404 via a mouse click that may create an additional outer control point 406c. FIG. 4C illustrates an example of adjusting the outline 404 so as to include an area 414 via a mouse click that may create an additional inner control point 406d. FIG. 4B and FIG. 4C also illustrate examples of representing the outline 404 with a dashed line (e.g., as a preview of the to-be-adjusted outline) in response to a user maintaining a cursor or a finger at a location for a certain time duration, as described above.

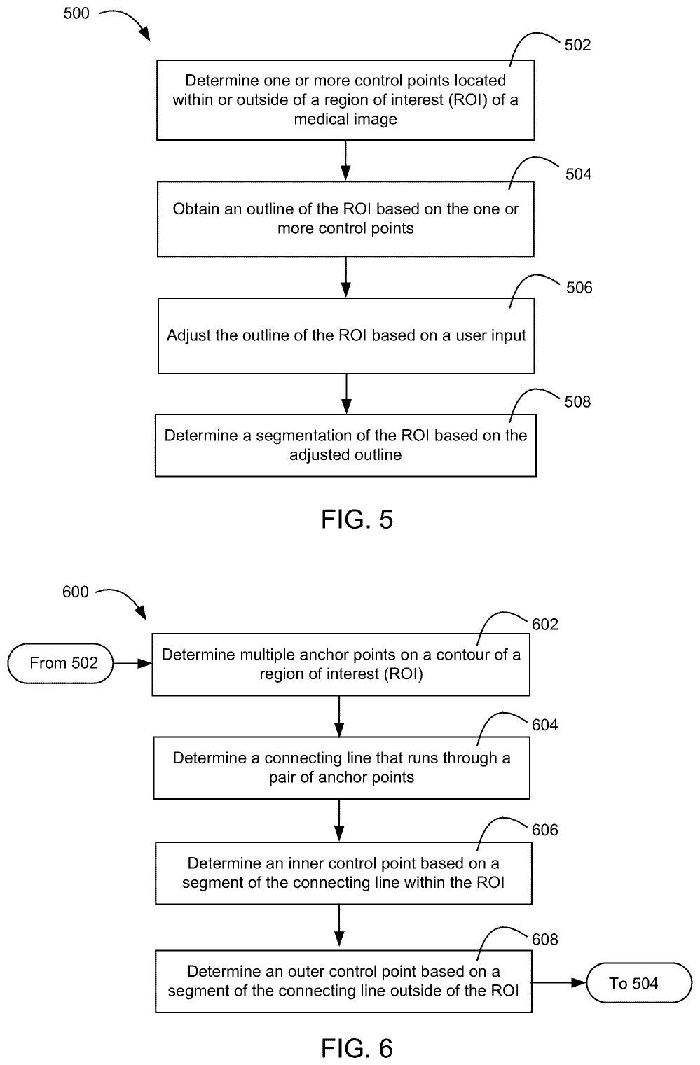

FIG. 5 shows a flow diagram illustrating an example method 500 for automatic segmentation of a medical image (e.g., medical image 100 of FIG. 1A), according to embodiments described herein. As shown in FIG. 5, an apparatus (e.g., the image segmentation apparatus described herein) may determine, at 502, one or more control points located within or outside of a region of interest (ROI) (e.g., ROI 102 of FIG. 1A) of a medical image (e.g., medical image 100). The one or more control points may include, for example, one or more inner control points that may be located within the ROI and the one or more outer control points that may be located outside of the ROI. At 504, the apparatus may obtain an outline of the ROI based on the one or more control points, and further adjust the outline of the ROI at 506 based on a user input. Using the adjusted outline, the apparatus may determine a segmentation of the ROI at 508, which may distinguish the portion of the medical image corresponding to the ROI from the rest of the medical image, for example, via color-coding.

FIG. 6 shows a flow diagram illustrating an example method 600 for generating one or more control points as part of operation 502 of method 500. As shown in FIG. 6, the method 600 may include determining multiple anchor points on a contour of an ROI at 602, wherein the contour of the ROI may be obtained based on a computer-generated segmentation of the ROI or a manual segmentation of the ROI (e.g., which may be received via a graphical user interface as described herein). The method 600 may further include determining a connecting line at 604 that may run through a pair of the anchor points, and further determining an inner control point at 604 based on a segment of the connecting line that is located within the ROI. Additionally, the method 600 may include determining an outer control point at 606 based on a segment of the connecting line that is located outside of the ROI. As described herein, the number of inner control points and the number of the outer control points to be generated may be configurable, and therefore the operations at 604 and/or 606 may be repeated according to the configuration, before the method 600 returns to operation 504 of FIG. 5.

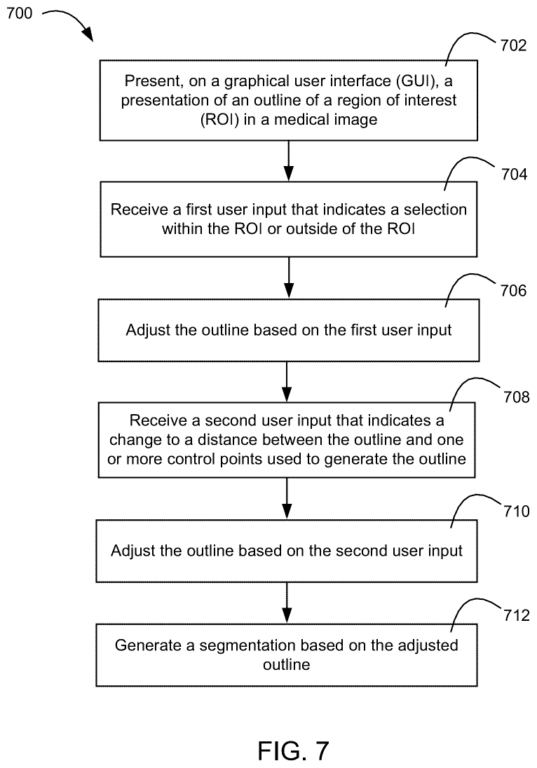

FIG. 7 shows a flow diagram illustrating an example method 700 for receiving a user input via a graphical user interface (GUI) and adjusting an outline of an ROI based on the user input. As shown in FIG. 7, the method 700 may include providing, on a graphical user interface (GUI), a presentation of an outline of an ROI in a medical image, wherein the outline may be generated based on one or more control points (e.g., one or more inner control points and one or more outer control points as described herein). The method 700 may further include receiving a first user input at 704 that indicates a selection of an area or a spot within the ROI or outside of the ROI. The selection may be made by a user, for example, via a mouse click on a computer screen or a tap on a touch screen device. In response to such as a selection, the method 700 may further include adjusting the outline at 706 by adding an area that corresponds to the selected area or spot to the outline, or by removing an area that corresponds to the selected area or spot from the outline.

In examples, the method 700 may additionally include receiving a second user input (e.g., the input received via element 212 of FIG. 2A) at 708 that may indicate a change to a distance between the outline and the one or more control points used to generate the outline. The distance may be, for example, a threshold distance (e.g., a minimum distance) between the outline and the one or more control points. In response to receiving such a threshold distance value, the outline may be adjusted at 710, for example, by expanding or shrinking the outline to satisfy the value indicated by the second user input. Once the outline has been adjusted (e.g., based on the first and second user inputs), a segmentation of the ROI may be generated at 712, for example, by marking pixels of the medical image that are within the outline as belonging to the ROI, and pixels outside of the ROI as belonging to the background of the medical image or another object in the medical image.

For simplicity of explanation, the operations associated with the methods (e.g., methods 500, 600, and/or 700) may be depicted and described herein with a specific orders. It should be appreciated, however, that the operations may occur in different orders, concurrently and/or with other operations not presented or described herein. Furthermore, it should be noted that not all operations illustrated may be included in the methods disclosed herein, and not all illustrated operations are required to be performed.

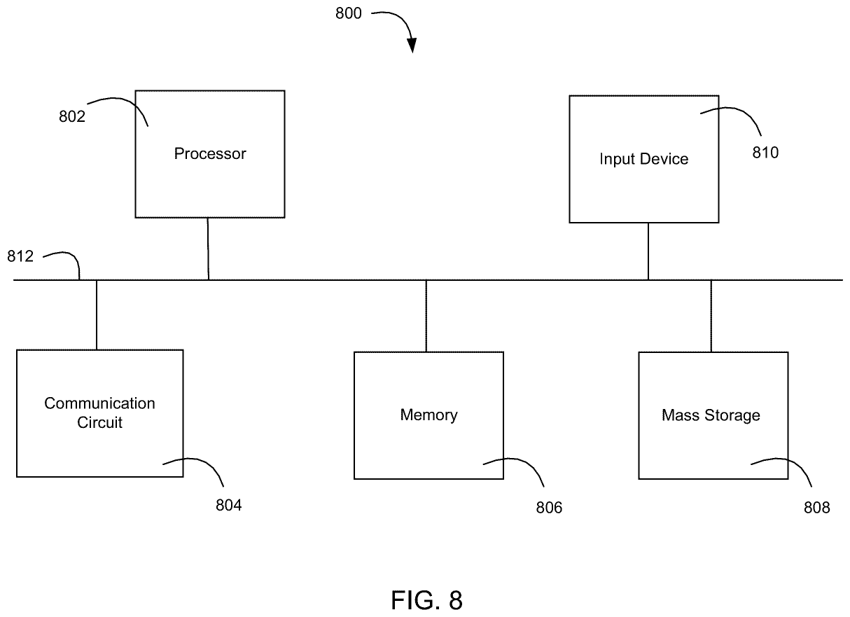

The systems, methods, and/or instrumentalities described herein may be implemented using one or more processors, one or more storage devices, and/or other suitable accessory devices such as display devices, communication devices, input/output devices, etc. FIG. 8 illustrates an example apparatus 800 that may be configured to perform the tasks described herein. As shown, the apparatus 800 may include a processor (e.g., one or more processors) 802, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. The apparatus 800 may further include a communication circuit 804, a memory 806, a mass storage device 808, an input device 810, and/or a communication link 812 (e.g., a communication bus) over which the one or more components shown in the figure may exchange information.

The communication circuit 804 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). The memory 806 may include a storage medium (e.g., a non-transitory storage medium) configured to store machine-readable instructions that, when executed, cause the processor 802 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. The mass storage device 808 may include one or more magnetic disks such as one or more internal hard disks, one or more removable disks, one or more magneto-optical disks, one or more CD-ROM or DVD-ROM disks, etc., on which instructions and/or data may be stored to facilitate the operation of the processor 802. The input device 810 may include a keyboard, a mouse, a voice-controlled input device, a touch sensitive input device (e.g., a touch screen), and/or the like for receiving user inputs to the apparatus 800.

It should be noted that the apparatus 800 may operate as a standalone device or may be connected (e.g., networked, or clustered) with other computation devices to perform the functions described herein. And even though only one instance of each component is shown in FIG. 8, a skilled person in the art will understand that the apparatus 800 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
a processor configured to:
obtain an outline of a region of interest (ROI) in a medical image, wherein the outline is obtained based on one or more control points located within the ROI or outside of the ROI;
adjust the outline of the ROI based on a user input, wherein the user input indicates a selection within the outline or outside of the outline, or a change to a distance between the outline and the one or more control points; and
determine a segmentation of the ROI based on the adjusted outline of the ROI.

2. The apparatus of claim 1, wherein the processor being configured to determine the outline of the ROI in the medical image comprises the processor being configured to:
determine multiple anchor points associated with the ROI;
determine, based on the multiple anchor points, one or more inner control points located within the ROI and one or more outer control points located outside of the ROI; and
obtain the outline of the ROI based on the one or more inner control points and the one or more outer control points.

3. The apparatus of claim 2, wherein the processor being configured to determine the multiple anchor points associated with the ROI comprises the processor being configured to obtain a segmentation mask associated with the ROI and select multiple points on a contour of the segmentation mask as the anchor points.

4. The apparatus of claim 3, wherein the segmentation mask is generated based on human annotation or a machine learning model.

5. The apparatus of claim 2, wherein the processor being configured to determine the one or more inner control points and the one or more outer control points based on the multiple anchor points comprises the processor being configured to:
determine a connecting line that runs through a pair of anchor points;
select a point on the connecting line that is within the ROI as an inner control point; and
select a point on the connecting line that is outside of the ROI as an outer control point.

6. The apparatus of claim 2, wherein the outline of the ROI is located between the one or more inner control points and the one or more outer control points, and wherein the processor is configured to determine the outline based on a predetermined value that indicates a proximity of the outline to the one or more inner control points or the one or more outer control points.

7. The apparatus of claim 1, wherein the one or more control points include a single inner control located within the ROI and multiple outer control points located outside of the ROI.

8. The apparatus of claim 1, wherein the processor being configured to adjust the outline of the ROI based on the user input comprises the processor being configured to:
determine whether the selection indicated by the user input is within the outline or outside of the outline;
based on a determination that the selection is within the outline, adjust the outline to exclude an area corresponding to the selection; and
based on a determination that the selected area or spot is outside of the outline, adjust the outline to include an area corresponding to the selection.

9. The apparatus of claim 1, wherein the processor is further configured to provide a graphical user interface (GUI) element for changing an area surrounded by the outline, and wherein the processor being configured to adjust the outline based on the user input comprises the processor being configured to receive the user input via the GUI element, determine a value of the area surrounded by the outline based on the user input, and adjust the outline of the ROI based on the determined value.

10. The apparatus of claim 1, wherein the processor being configured to adjust the outline based on the user input comprises the processor being configured to provide a preview of the adjustment to be made to the outline and adjust the outline in response to receiving a confirmation of the adjustment.

11. A method for annotating a region of interest (ROI) in a medical image, the method comprising:
obtaining an outline of the ROI in the medical image, wherein the outline is obtained based on one or more control points located within the ROI or outside of the ROI;
adjusting the outline of the ROI based on a user input, wherein the user input indicates a selection within the outline or outside of the outline, or a change to a distance between the outline and the one or more control points; and
determining a segmentation of the ROI based on the adjusted outline of the ROI.

12. The method of claim 11, wherein determining the outline of the ROI in the medical image comprises:
determining multiple anchor points associated with the ROI;
determining, based on the multiple anchor points, one or more inner control points located within the ROI and one or more outer control points located outside of the ROI; and
obtaining the outline of the ROI based on the one or more inner control points and the one or more outer control points.

13. The method of claim 12, wherein determining the multiple anchor points associated with the ROI comprises obtaining a segmentation mask associated with the ROI and selecting multiple points on a contour of the segmentation mask as the anchor points, and wherein the segmentation mask is generated based on human annotation or a machine learning model.

14. The method of claim 12, wherein determining the one or more inner control points and the one or more outer control points based on the multiple anchor points comprises:
determining a connecting line that runs through a pair of anchor points;
selecting a point on the connecting line that is within the ROI as an inner control point; and
selecting a point on the connecting line that is outside of the ROI as an outer control point.

15. The method of claim 12, wherein the outline of the ROI is located between the one or more inner control points and the one or more outer control points, and wherein obtaining the outline of the ROI comprises determining the outline based on a predetermined value that indicates a proximity of the outline to the one or more inner control points or the one or more outer control points.

16. The method of claim 11, wherein the one or more control points include a single inner control located within the ROI and multiple outer control points located outside of the ROI.

17. The method of claim 11, wherein adjusting the outline of the ROI based on the user input comprises:
determining whether the selection indicated by the user input is within the outline or outside of the outline;
based on a determination that the selection is within the outline, adjusting the outline to exclude an area corresponding to the selection; and
based on a determination that the selected area or spot is outside of the outline, adjusting the outline to include an area corresponding to the selection.

18. The method of claim 11, further comprising providing a graphical user interface (GUI) element for changing an area surrounded by the outline and the one or more control points, wherein adjusting the outline based on the user input comprises:
receiving the user input via the GUI element;
determining a value of the area surrounded by the outline based on the user input; and
adjusting the outline of the ROI based on the determined value.

19. The method of claim 11, wherein adjusting the outline based on the user input comprises providing a preview of the adjustment to be made to the outline and adjusting the outline in response to receiving a confirmation of the adjustment.

20. A non-transitory computer-readable medium comprising instructions that, when executed by a processor included in a computing device, cause the processor to implement the method of claim 11.
